(19) United States
(12) Patent Application Publication
(10) Pub. No.: US 2023/0221303 A1
(43) Pub. Date: Jul. 13, 2023

(54) POTENCY ASSAY

(71) Applicant: Longeveron Inc., Miami, FL (US)

(72) Inventors: Anthony A. Oliva, Miami, FL (US); Ben Hitchinson, Miami, FL (US)

(73) Assignee: Longeveron Inc., Miami, FL (US)

(21) Appl. No.: 17/996,529

(22) PCT Filed: Apr. 20, 2021
(86) PCT No.: PCT/US2021/028202

§ 371 Date: Oct. 19, 2022

Related U.S. Application Data
(60) Provisional application No. 63/012,884, filed on Apr. 20, 2020.

(51) Int. Cl. G01N 33/50; G01N 33/68

(52) U.S. Cl. CPC G01N 33/5073; G01N 33/6869; G01N 2333/525; G01N 2333/54

(57) ABSTRACT

A method for assessing the potency of MSCs to produce anti-inflammatory cytokines in response to a pro-inflammatory stimulus. The method comprises stimulating the MSCs with one or more proinflammatory cytokines, such as TNF-α, for a duration of time and then identifying and quantifying the production of anti-inflammatory cytokines. MSCs that produce potent levels of anti-inflammatory cytokines in response to TNF-α can be used in treatments for aging-related conditions, including aging frailty and Alzheimer's disease, and can also be used to treat corona virus infections. The method shows that TNF-α induced MSCs robustly secrete several anti-inflammatory cytokines, including IL-1 receptor antagonist (IL-1RA), IL-10, and granulocyte colony stimulating factor (G-CSF).

### CROSS-REFERENCE TO RELATED APPLICATIONS

This application claims priority benefit of U.S. Provisional Patent Application No. 63/012,884, filed Apr. 20, 2020, the contents of which are incorporated herein in their entirety.

### FIELD

Provided herein are methods for assessing the potency of human mesenchymal stem cells to produce anti-inflammatory cytokines in response to being exposed to pro-inflammatory cytokines, such as TNF-α. The human mesenchymal stem cells that demonstrate adequate anti-inflammatory cytokine production can then be used in methods for the treatment of diseases that involve prolonged inflammation such as aging frailty, Alzheimer's disease and coronavirus infections.

### BACKGROUND

Aging frailty poses a very concerning problem for the overall health and well-being of individuals. Aging frailty is a geriatric syndrome characterized by weakness, low physical activity, slowed motor performance, exhaustion, and unintentional weight loss. See Yao, X. et al., Clinics in Geriatric Medicine 27(1):79-87 (2011). Furthermore, there are many studies showing a direct correlation between aging frailty and inflammation. See Hubbard, R. E. et al., Biogerontology 11(5):635-641 (2010).

Immunosenescence is characterized by a low grade, chronic systemic inflammatory state known as inflammaging. See Franceshi, C. et al., Annals of the New York Academy of Sciences 908:244-254 (2000). This heightened inflammatory state or chronic inflammation found in aging and aging frailty leads to immune dysregulation and a complex remodeling of both innate and adaptive immunity. In immunosenescence, the T cell and B cell repertoire is skewed resulting in an increase in CD8⁺ T effector memory cells re-expressing CD45ra (TEMRA) and in the CD19⁺ late/exhausted memory B cells, and a decrease in the CD8⁺ Naïve T cells, and in the switched memory B cells (CD27⁺). See Blomberg, B. B. et al., Immunologic Research 57(1-3):354-360 (2013); Colonna-Romano, G. et al., Mechanisms of Ageing and Development 130(10):681-690 (2009); and Koch S. et al., Immunity & Ageing: 5:6 (2008). This shift in the T cell and B cell repertoire results in a refractory or less efficient immune status. This deterioration of the immune system causes greater susceptibility to infectious diseases and reduced responses to vaccination. Optimal B cell function is critical for production of effective antibody responses to vaccines and protection from infectious agents. It is well known that age-associated increase in systemic inflammation (TNF-α, IL-6, IL-8, INFγ and CRP) induces impaired B cell function leading to poor antibody responses and decreased vaccine efficacy.

Inflammaging has received considerable attention because it proposes a link between immune changes and a number of diseases and conditions (such as aging frailty) common in old age. Circulating inflammatory mediators such as cytokines and acute phase proteins are markers of the low-grade inflammation observed to increase with aging. These pro-inflammatory cytokines (e.g., TNF-α, IL-6) impair the capacity of B cells to make protective antibodies to exogenous antigens and vaccines. This impaired B cell response is measured by reduced class switch recombination (CSR) which is the ability of immunoglobulins to switch isotype from IgM to a secondary isotype (IgG, IgA, or IgE). Immunoglobulin isotype switching is crucial for a proper immune response as the effector functions differ in each isotype. A key player in CSR and somatic hypermutation (SUM) is the enzyme, activation-induced cytidine deaminase (AID), encoded by the Aicda gene. AID's basic function in CSR and SUM is to initiate breaks in the DNA by converting cytosines to uracils in the switch and variable regions of immunoglobulins.

It has also been shown in humans that the amount of TNF-α made: (1) depends on the amount of system inflammation and (2) impairs the ability of the same B cells to be stimulated with mitogens or antigens. See Frasca, D. et al., Journal of Immunology 188(1):279-286 (2012). Thus, the immune response in subjects suffering from aging frailty is impaired for a number of reasons.

TNF-α expression is also involved in the initiation, maintenance, and amplification of the immune processes which produce neurologic inflammation, and which have been implicated in the pathogenesis of Alzheimer's disease and related dementias, and other forms of inflammation resulting in neurological damage.

Alzheimer's disease (AD) is a chronic progressive neurodegenerative brain disease—syndrome of the aging. It is a major contributor to morbidity and modality in the elderly in nearly 5 million Americans. AD accounts for 70% of all cases of dementia. Dementia is a huge public health concern, with a new case diagnosed somewhere in the world every 7 seconds. There is no cure for the disease, which worsens as it progresses, and eventually leads to death within 7 years. Less than three percent of individuals live more than fourteen years after diagnosis. People diagnosed as having AD are usually over 65 years of age and have challenges completing standard verbal and visual memory tests, in addition to decision-making and problem-solving tasks. In 2006, there were 26.6 million sufferers worldwide and 5 million of them in the USA. Alzheimer's disease is predicted to affect 1 in 85 people globally by 2050. Early symptoms often erroneously thought to be ‘age-related’ concerns, or manifestations of stress.

Alzheimer' s disease (AD) involves complex pathology and encompassing diverse mechanisms in addition to β-amyloid deposition and neurofibrillary tangles. There is growing recognition that a pro-inflammatory state contributes to the ensuing dementia. In this regard, proinflammatory cytokines are abundant in the vicinity of amyloid deposits and neurofibrillary tangles, and an association exists between systemic inflammation and β-amyloid accumulation. Furthermore, individuals can have significant amyloid deposits and neurofibrillary tangles at autopsy that would qualify for them for a diagnosis of AD, yet never showed a history of dementia: in these cases, expression of inflammatory markers was dramatically less than in AD patients.

AD is also characterized by impaired neurovasculature that contributes to adverse outcomes. Notable among these is hypoperfusion and compromise of the blood-brain barrier (BBB). Resulting compromise of the BBB can impair exchange across the endothelium. Impaired exchange across the endothelium appears in part due to direct inhibition of AβP on endothelial cell proliferation and migration. Ultimately, there is inefficient clearance of AβP across the BBB, and resultant accumulation of AβP in the brain parenchyma. Thus, the compromised neurovasculature is another important therapeutic target in AD.

Coronavirus infections have been shown to be a significant threat to the human population. Specifically, patients infected with COVID-19 suffer from especially poor outcomes if they require advanced respiratory support. The mortality rate of these patients reaches about 54%. Clinical deterioration often occurs 7-10 days after the onset of symptoms, in association with declining viral titres, suggesting that pathology is driven by inflammation rather than direct viral injury. Inflammatory markers are often substantially elevated in patients with severe COVID-19, thereby causing a hyperinflammatory syndrome which might contribute to morbidity and mortality of the infection. Hyperinflammatory syndrome typically involves uncontrolled, self-perpetuating, and tissue-damaging inflammatory activity.

Diseases similar to or recited above are typically treated using therapeutic agents such as small molecules, proteins, vaccines or antibodies. The use of cell therapies to treat the above diseases is not well-documented or explored within the art. Cell therapies represent a new and exciting treatment modality across a wide range of therapeutic indications.

Mesenchymal stem cells are multipotent cells able to migrate to sites of injury, while also being immunoprivileged by not detectably expressing major histocompatibility complex class II (MHC-II) molecules, and expressing MHC-I molecules at low levels. See Le Blanc, K. et al., Lancet 371(9624):1579-1586 (2008) and Klyushnenkova E. et al., J. Biomed. Sci. 12(1):47-57 (2005). As such, allogeneic mesenchymal stem cells hold great promise for therapeutic and regenerative medicine, and have been repeatedly shown to have a high safety and efficacy profile in clinical trials for multiple disease processes. See Hare, J. M. et al., Journal of the American College of Cardiology 54(24):2277-2286 (2009); Hare, J. M. et al., Tex. Heart Inst. J. 36(2):145-147 (2009); and Lalu, M. M. et al., PloS One 7(10):e47559 (2012). They have also been shown to not undergo malignant transformation after transplantation into patients. See Togel F. et al., American Journal of Physiology Renal Physiology 289(1):F31-F42 (2005). Treatment with mesenchymal stem cells has been shown to ameliorate severe graft-versus-host disease, protect against ischemic acute renal failure, contribute to pancreatic islet and renal glomerular repair in diabetes, reverse fulimant hepatic failure, regenerate damaged lung tissue, attenuate sepsis, and reverse remodeling and improve cardiac function after myocardial infarction. See Le Blanc K. et al., Lancet 371(9624):1579-1586 (2008); Hare, J. M. et al., Journal of the American College of Cardiology 54(24):2277-2286 (2009); Togel F. et al., American Journal of Physiology Renal Physiology 289(1):F31-F42 (2005); Lee R. H. et al., PNAS 103(46):17438-17442 (2006); Parekkadan, B. et al., PloS One 2(9):e941 (2007); Ishizawa K. et al., FEBS Letters 556(1-3):249-252 (2004); Nemeth K. et al., Nature Medicine 15(1):42-49 (2009); Iso Y. et al., Biochem. Biophys. Res. Comm. 354(3):700-706 (2007); Schuleri K. H. et al., Eur. Hearth J. 30(22):2722-2732 (2009); and Heldman A. W. et al., JAMA 311(1):62-73 (2014). Furthermore, mesenchymal stem cells are also a potential source of multiple cell types for use in tissue engineering. See Gong Z. et al., Methods in Mol. Bio. 698:279-294 (2011); Price, A. P. et al., Tissue Engineering Part A 16(8):2581-2591 (2010); and Togel F. et al., Organogenesis 7(2):96-100 (2011).

Mesenchymal stem cells have immuno-modulatory capacity. They control inflammation and the cytokine production of lymphocytes and myeloid-derived immune cells without evidence of immunosuppressive toxicity and are hypo-immunogenic. See Bernardo M. E. et al., Cell Stem Cell 13(4):392-402 (2013).

In vivo studies have shown that human mesenchymal stem cells undergo site-specific differentiation into various cell types, including myocytes and cardiomyocytes, when transplanted into fetal sheep. See Airey J. A. et al., Circulation 109(11):1401-1407 (2004). These mesenchymal stem cells can persist for as long as 13 months in multiple tissues after transplantation in non-immunosuppressed immunocompetent hosts. Other in vivo studies using rodents, dogs, goats, and baboons similarly demonstrate that human mesenchymal stem cells xenografts do not evoke lymphocyte proliferation or systemic allo-antibody production in the recipient. See Klyushnenkova E. et al., J. Biomed. Sci. 12(1):47-57 (2005); Aggarwal S. et al., Blood 105(4):1815-22 (2005); Augello A. et al., Arthritis and Rheumatism 56(4):1175-86 (2007); Bartholomew A. et al., Exp Hematol. 30(1):42-48. (2002); Dokic J. et al., European Journal of Immunology 43(7):1862-72 (2013); Gerdoni E. et al., Annals of Neurology 61(3):219-227 (2007); Lee S. H. et al., Respiratory Research 11:16 (2010); Urban V. S. et al., Stem Cells 26(1):244-253 (2008); Yang H. et al., PloS One 8(7):e69129 (2013); Zappia E. et al., Blood 106(5):1755-1761 (2005); Bonfield T. L. et al., American Journal of Physiology Lung Cellular and Molecular Physiology 299(6):L760-70 (2010); Glenn J. D. et al., World Journal of Stem Cells. 6(5):526-39 (2014); Guo K. et al., Frontiers in Cell and Developmental Biology 2:8 (2014); Puissant B. et al., British Journal of Haematology 129(1):118-129 (2005); and Sun L. et al., Stem Cells 27(6):1421-32 (2009). Taken as a whole, these repeated finding of allogeneic safety and efficacy solidify the notion for using mesenchymal stem cells as an allograft for successful tissue regeneration.

AD animal model studies have also been shown to support the clinical potential of MSCs. See Neves AF et al., Exp. Neurol. 2021:113706. The beneficial effects include decreasing inflammation, increasing Aβ-degrading factors and Aβ clearance, decreasing hyperphosphorylated tau, and elevating alternatively activated (M2) microglial markers. These benefits appear, at least in part, due to Aβ-induced MSC release of chemoattractants that recruit alternative microglia into the brain to reduce Aβ deposition. See Lee J K et al., Stem Cells 2012; 30(7):1544-55. MSCs are effective in young AD-model mice prior to Aβ accumulations, leading to significant decreases in cerebral Aβ deposition, and a significant increase in expression of pre-synaptic proteins. See Bae J S et al., Curr Alzheimer Res. 2013;10(5):524-31. Impressively, these effects were sustained for at least 2 months, and suggest MSCs could be useful as an interventional therapeutic in prodromal AD. In short, AD preclinical studies have shown that MSCs can cross the BBB, inhibit neuroinflammation, promote neurogenesis, inhibit β-amyloid deposition and promote clearance, reduce apoptosis, promote hippocampal neurogenesis, improve dendritic morphology, and improve behavioral and spatial memory performance

### SUMMARY

The property of mesenchymal stem cells (MSCs) to produce immunomodulatory cytokines in response to a pro-inflammatory stimulus is an important therapeutic mechanism of action employed by MSCs.

Accurate, reproducible, and relevant assays for assessing potency of cells used in cell therapies are important for quality control purposes, for example, ensuring stability and consistency of cell-based therapeutic products.

Current assays used in the art for assessing the potency of cells focus on identifying the expression of specific biomarkers or cell surface receptors. These assays are expected to provide indirect measurements on the potency of cells (e.g., MSCs expressing TNFR1 are expected to inhibit PBMC proliferation). Thus the “potency assays” used within the art are identity assays that measure the expression of cell receptors or biomarkers and fail to accurately measure the ability or potency of a cell to express or produce key macromolecules, such as anti-inflammatory cytokines.

MSC potency assays have been developed wherein the MSCs are stimulated with LPS; however, these potency assays produce “irrelevant” stimulations (e.g., irrelevant because LPS stimulation is a mimic of bacterial infection and MSCs are not used as an anti-bacterial agent). These assays are further irrelevant since MSCs generally do not express TLR4 or CD14, which are both required for LPS stimulation and signaling. Hence, an objective of the present application is to provide a potency assay that accurately determines the ability of mesenchymal stem cells (MSC) to produce immunomodulatory cytokines in response to pro-inflammatory cytokines, such as TNF-α. Ideally, measurements are made for physiologically meaningful components.

Provided herein are methods for assessing MSC potency, e.g., assessing the potency of MSCs in a preparation of cells (e.g., a preparation of MSCs that belong to a lot of cells intended for therapeutic use). The methods provided herein utilize a TNF-α stimulation step, prior to assessing cell or cell lot potency, to assess whether the MSCs produce anti-inflammatory cytokines, and to what level, as compared to standard cell potency assays used in the art which only involve detecting the presence of cell surface receptors or biomarkers and fail to assess whether the cells are capable of expressing the molecules associated with stimulation of said receptors or biomarkers. It has been determined that incorporating the TNF-α stimulation step results in potency assay with increased reliability and decreased variability across MSC preparations taken from same cell lot, as well as MSC preparations comprising the same cell type but taken from different cell lots.

### BRIEF DESCRIPTION OF DRAWINGS

FIG. 1 depicts the production levels of anti-inflammatory cytokines after stimulation of MSCs with recombinant human TNFα.

FIG. 2 depicts the viability of MSCs after stimulation with recombinant human TNFα.

FIG. 3 depicts the production levels of anti-inflammatory cytokines after stimulation of MSCs with recombinant human TNFα over 24 hours.

FIG. 4 depicts the production levels of IL-8 and IL-13 after MSCs were sensitized to IL-17A stimulation but exposure to recombinant human TNFα for 1 hour.

### DETAILED DESCRIPTION

One aspect of the present application relates to a method for assessing the potency of MSCs to produce anti-inflammatory cytokines.

In one embodiment, the method comprises stimulating the MSCs with a pro-inflammatory cytokine or molecule for a duration of time before identifying and quantifying the levels of anti-inflammatory cytokine production.

The MSCs can be derived from bone marrow, adipose tissue, peripheral blood, a lung, a heart, amniotic fluid, inner organs, an amniotic membrane, an umbilical cord or a placenta, or other tissue, or differentiated from induced pluripotent stem cells (IPSCs) or other sources.

The MSCs can be stimulated with a pro-inflammatory cytokines or molecules. The pro-inflammatory cytokines can be selected from TNF-α, IL-1, IL-2, IL-6, IL-12, IL-17A, IL-18, IFN-γ or any combination thereof. In some embodiments, the MSCs are stimulated with both TNF-α and IL-17A, or other combinations. Other pro-inflammatory molecules include C-reactive protein (CRP) or virulence factors. Virulence factors can be any viral molecule that aids in the colonization of a niche in a host, immunoevasion or evasion of a host's immune response, immunosuppression or inhibition of a host's immune response, entry into and exit out of cells or obtaining nutrition from a host. One example of a virulence factor is a SARS-CoV-2 spike protein.

Surprisingly, MSCs have been shown to not produce or produce significantly lower levels of anti-inflammatory cytokines when treated with IL-17A alone. When MSCs are treated with IL-17A and TNF-α together, the MSCs produce significantly higher levels of anti-inflammatory cytokines. The importance of this unexpected discovery stems from the current criteria required to assess the potency of cells, which is to confirm that the cells express certain receptors or biomarkers without assessing the receptors' abilities to promote production of specific molecules. This discovery confirms that even though a cell possesses a receptor known to produce specific molecules, the cell may not produce said molecules at an efficient potency to be useful in following treatments. Furthermore, it shows that MSCs can respond differently to different combinations of pro-inflammatory molecules that are indication- or patient-specific, to best suit particular treatments to specific patients.

The amount of the pro-inflammatory cytokine or molecule used to stimulate the MSCs can range from 10 fg/mL to 10 μg/mL, 1 pg/mL to 10 μg/mL, 1 μg/mL to 10 μg/mL, 1 fg/mL to 1 pg/mL, 1 fg/mL to 10 μg/mL or 1 pg/mL to 5 μg/mL, under conditions where 500 to 50,000 MSCs are cultured in 50 to 200 microliters of medium. Concentrations are scaled accordingly with changes in cell number and/or volume.

The MSCs can be stimulated with a pro-inflammatory cytokine or molecule for between 1 hour to 24 hours, 1 hour to 12 hours, 2 hours to 6 hours or 1 hour to 4 hours, 24 hours to 120 hours, 24 hours to 72 hours or more than 120 hours before quantifying the levels of anti-inflammatory cytokine production.

The anti-inflammatory cytokines that can be examined and quantified after stimulation of the MSCs with a pro-inflammatory cytokine or molecule are IL-1RA, IL-4, IL-7, IL-8, IL-10, IL-13, G-CSF or any combination thereof.

In other embodiments, stimulation of the MSCs with a pro-inflammatory cytokine or molecule may lead to the production of an anti-inflammatory molecule in concentrations ranging from 1 fg/mL to 100 ng/mL, 1 fg/mL to 10 μg/mL, 1 fg/mL to 10 pg/mL, 1 fg/mL to 10 fg/mL, 10 fg/mL to 10 pg/mL, 10 pg/mL to 10 μg/mL, 10 μg/mL to 1 mg/mL, 1 pg/mL to 10 pg/mL, 1 μg/mL to 10 μg/mL or 10 pg/mL to 1 μg/mL per every 500 to 50,000 cells cultured in 50 to 200 microliters of medium. Concentrations may be scaled accordingly with changes in cell number and/or volume of medium.

In some embodiments, the method further comprises checking for the expression of biomarkers on the MSCs before stimulation with a pro-inflammatory cytokine. The biomarkers that can be searched for include CD105⁺, CD90⁺, CD73⁺, CD45⁻, CD34⁻, CD19⁻, CD11b⁻, HLA-DR⁻, IL-17RA⁺ or any combination thereof.

In other embodiments, the method can further comprise a step of seeding the MSCs onto a substrate before stimulation with a pro-inflammatory cytokine. The substrate can be a membrane, a plastic surface, a glass surface or a cell culture well plate, such as a 96-well plate, with or without an added substrate coating. The duration for seeding the MSCs onto a substrate can be from 1 hour to 24 hours, 1 hour to 12 hours, 2 hours to 6 hours or 1 hour to 4 hours. The MSCs should be properly adhered onto the substrate after the seeding duration has passed.

The MSCs can be divided into smaller populations of MSCs before stimulation with a pro-inflammatory cytokine. Separation of the MSCs into smaller populations provides a more accurate assessment of the MSCs ability to produce anti-inflammatory cytokines after stimulation.

In some embodiments, the method can further comprise a step of isolating the supernatants of the MSCs after stimulation with a pro-inflammatory cytokine. The supernatants can be stored at −80° C. once they have been collected. The supernatants can be further analyzed to determine the levels of anti-inflammatory cytokines produced from the MSCs through the use of electrochemiluminescence immunoassays. Methods of detection typically used in potency assays are not as sensitive as electrochemiluminescence immunoassays, so the use of electrochemiluminescence immunoassays allows the detection of cytokines in femtogram concentrations produced by MSCs.

In other embodiments, the method further comprises performing a viability assay on the MSCs after they have been stimulated with a pro-inflammatory cytokine for a duration of time. The viability assay can be an ATP detection assay such as CellTiter-Glo assay (Promega), a tetrazolium reduction assay, a resazurin reduction assay, a protease viability marker assay, a sodium-potassium ratio assay, a cytolysis or membrane leakage assay, a mitochondrial activity or caspase assay, a functional assay, a genomic and proteomic assay or any combination thereof. The viability of the MSCs can also be assessed through the use of flow cytometry.

The viability of the MSCs after stimulation with a pro-inflammatory cytokine may be greater than 70% when compared to MSC populations treated with a vehicle.

In other embodiments, the method further comprises assigning a grade to the potency of the MSCs based on the amount of produced anti-inflammatory molecules. The grades assigned to the potency of the MSCs include thresholds grades wherein the MSCs may possess a potency grade of producing at least 1 fg/mL to 100 ng/mL, 1 fg/mL to 10 μg/mL, 1 fg/mL to 10 pg/mL, 1 fg/mL to 10 fg/mL, 10 fg/mL to 10 pg/mL, 10 pg/mL to 10 μg/mL, 10 μg/mL to 1 mg/mL, 1 pg/mL to 10 pg/mL, 1 μg/mL to 10 μg/mL or 10 pg/mL to 1 μg/mL of anti-inflammatory cytokines per every 500 to 50,000 cells cultured in 50 to 200 microliters of medium.

### EXAMPLES

### Example 1

A population of human MSCs derived from bone marrow aspirates and subsequently cryopreserved were thawed. Upon thaw, an aliquot of the MSCs were taken for immunophenotyping to confirm cell identity. This included confirming that the MSCs expressed CD105, CD90 and CD73 but lacked expression of CD45, CD34, CD19, CD11b and HLA-DR.

From the remaining cells, 10,000 MSCs were seeded into wells of a 96 well plate and allowed to adhere overnight in culture medium. The following day, media in the 96-well plate was replaced with fresh culture medium and either vehicle (PBS, Gibco) or concentrations of pro-inflammatory cytokines (R&D Systems). After 24 hours, supernatants were collected and cell viability was assessed using a Cell-titer glo assay. Supernatants were analyzed for immunomodulatory cytokine production by MSD electrochemiluminescence immunoassays. The supernatants were incubated on appropriate MSD plates overnight at 4° C., before detection the following day.

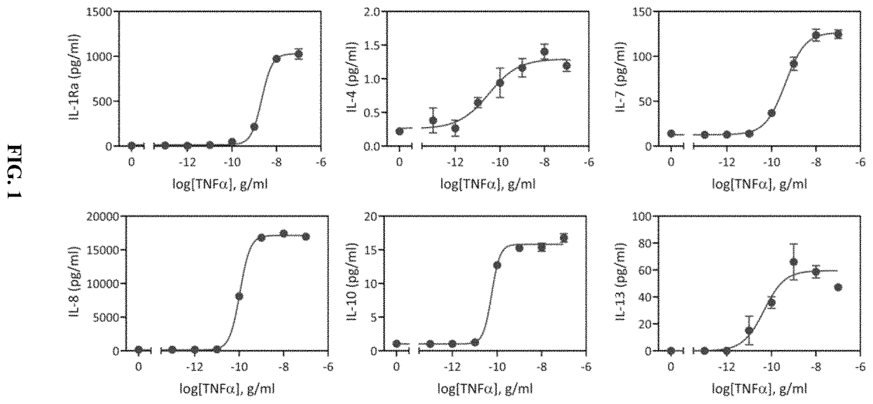

FIG. 1 depicts the concentration levels of the immunomodulatory cytokine produced from the MSCs in the supernatants after stimulation with TNF-α for 24 hours. Data shown is mean±standard deviation of a representative experiment from 3 individual lots of MSCs. The MSCs showed robust production of multiple immunomodulatory cytokines including IL-1RA, IL-4, IL-7, IL-8, IL-10 and IL-13 within 24 hours of stimulation with TNF-α in a dose-dependent manner.

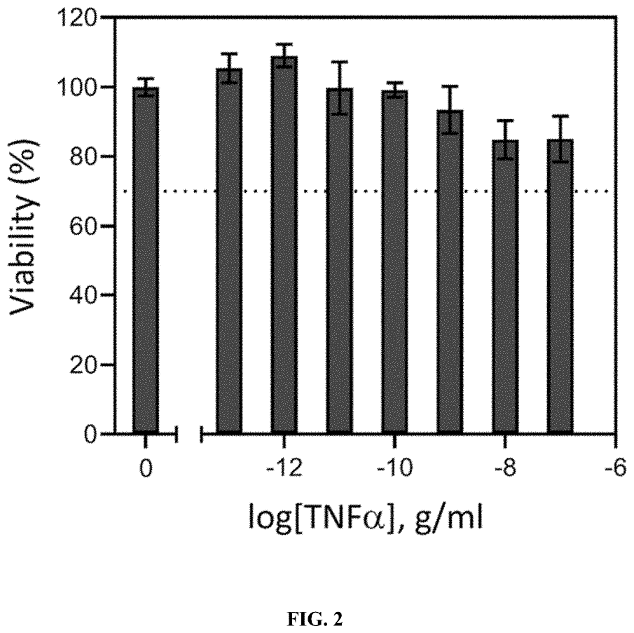

FIG. 2 depicts the cell viability of the MSCs after incubation with TNF-α for 24 hours. Supernatants were collected and a cell titer glo reagent was added to the MSCs. The reagent was allowed to incubate for 10 minutes at room temperature. After 10 minutes, luminescence readings were taken on a SpectraMax plate reader. Cell viability was determined by normalizing values to cells treated with vehicle only. All MSCs treated with TNF-α, including those stimulated with the highest concentration of 100 ng/ml, showed mean cell viability of above 80%.

To measure the production of immunomodulatory cytokines by MSCs over time, 10,000 MSCs from Example 1 were seeded per well into a 96 well plate in culture medium and allowed to adhere overnight. The following day, media was replaced with fresh culture medium, and cells were stimulated for the indicated amount of time with either vehicle (PBS, Gibco) or 10 pg/ml of recombinant human TNF-α (R&D Systems). Supernatants were collected and analyzed for anti-inflammatory cytokine production through MSD electrochemiluminescence immunoassays.

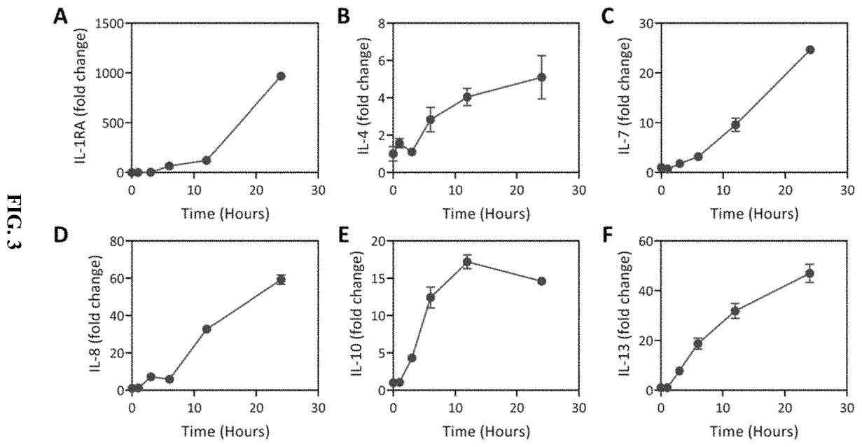

FIG. 3 shows the anti-inflammatory cytokine production of the MSCs after exposure to 10 pg/mL of TNF-α at various time points. Data shown as mean fold change ±SD of a representative experiment from 3 individual lots of MSCs. The cells showed sustained production of IL-1RA, IL-4, IL-7, IL-8, IL-10 and IL-13 over the 24 hour timecourse.

### Example 3

To measure the production of immunomodulatory cytokines by MSCs in response to IL-17A, 10,000 LMSCs were seeded per well into a 96 well plate in culture medium and allowed to adhere overnight. The following day, media was replaced with fresh culture medium, and cells were stimulated for one hour with either vehicle (PBS, Gibco) or 1 pg/ml of recombinant human TNF-α (R&D Systems) prior to the addition of the indicated concentrations of IL-17A for 24 hours. Supernatants were collected and analyzed for anti-inflammatory cytokine production.

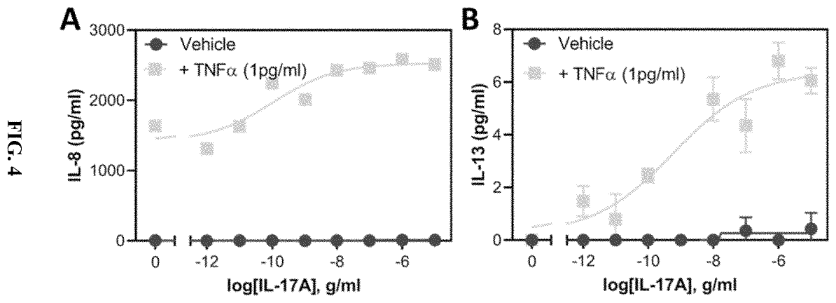

FIG. 4 depicts that production of anti-inflammatory cytokines IL-8 and IL-13 after exposure of IL-17A alone or IL-17A and TNF-α. The cells showed no or minimal production of IL-8 and IL-13 when stimulated with IL-17A alone (FIG. 4a), but when exposed to IL-17A and TNF-α, IL-8 and IL-13 production significantly increased in a dose dependent response to IL-17A, suggesting TNF-α sensitizes MSCs to IL-17A. These results were also discovered during examination of IL-13 production (FIG. 4b).

The present disclosure is not to be limited in scope by the specific embodiments described herein. Indeed, various modifications of the subject matter provided herein, in addition to those described, will become apparent to those skilled in the art from the foregoing description. Such modifications are intended to fall within the scope of the appended claims.

Various publications, patents and patent applications are cited herein, the disclosures of which are incorporated by reference in their entireties.

We claim:

1. A method for assessing the potency of human mesenchymal stem cells (MSCs), comprising:
stimulating a population of MSCs with a pro-inflammatory cytokine or other pro-inflammatory molecule;
identifying anti-inflammatory cytokine production from said MSCs; and
quantifying levels of the anti-inflammatory cytokine production from said MSCs.

2. The method according to claim 1, wherein the pro-inflammatory cytokine is TNF-α, IL-17a or a combination thereof.

3. The method according to claim 1, wherein the pro-inflammatory cytokine is TNF-α.

4. The method according to claim 1, wherein the stimulation step occurs between 1 hour and 24 hours.

5. The method according to claim 1, wherein the pro-inflammatory cytokine is administered to the MSCs in an amount ranging from 0.1 pg/mL to 1 μg/mL.

6. The method according to claim 1, wherein the MSCs are derived from bone marrow, adipose tissue, peripheral blood, a lung, a heart, amniotic fluid, inner organs, an amniotic membrane, an umbilical cord or a placenta, or other tissue, or differentiated from induced pluripotent stem cells (IPSCs) or other sources.

7. The method according to claim 1, wherein the anti-inflammatory cytokines that can be identified and quantified are selected from the group consisting of IL-1RA, IL-4, IL-7, IL-8, IL-10, IL-13, G-CSF and combinations thereof.

8. The method according to claim 1, wherein the method further comprises a step of checking for the expression of biomarkers on the MSCs before stimulation with the pro-inflammatory cytokine.

9. The method according to claim 8, wherein the biomarkers that are searched for include CD105⁺, CD90⁺, CD73⁺, CD45⁻, CD34⁻, CD19⁻, CD11b⁻, IL-17RA⁺, HLA-DR⁺ or any combination thereof.

10. The method according to claim 1, wherein the method can further comprise a step of seeding the MSCs onto a substrate before stimulation with the pro-inflammatory cytokine.

11. The method according to claim 10, wherein the substrate is a membrane, a plastic surface, a glass surface or a cell culture well plate, such as a 96-well plate, with or without an added substrate coating.

12. The method according to claim 10, wherein the seeding of the MSCs onto the substrate lasts from 1 hour to 24 hours.

13. The method according to claim 1, wherein the MSCs are divided into smaller populations of MSCs before stimulation with the pro-inflammatory cytokine.

14. The method according to claim 1, wherein the method further comprises a step of isolating supernatants of the MSCs after stimulation with the pro-inflammatory cytokine.

15. The method according to claim 14, wherein the supernatants are cryopreserved once they have been isolated from the MSCs.

16. The method according to claim 14, wherein the supernatants are analyzed with a electrochemiluminescence immunoassay or other assays to determine the levels of anti-inflammatory cytokines produced by the MSCs.

17. The method according to claim 1, wherein the method further comprises performing a viability assay on the MSCs after they have been stimulated with the pro-inflammatory cytokine.

18. The method according to claim 17, wherein the viability assay is an ATP detection assay, a tetrazolium reduction assay, a resazurin reduction assay, a protease viability marker assay, a sodium-potassium ratio assay, a cytolysis or membrane leakage assay, a mitochondrial activity or caspase assay, a functional assay, a genomic and proteomic assay or any combination thereof.

19. The method according to claim 17, wherein the viability assay comprises the use of flow cytometry.

20. The method according to claim 17, wherein the viability of the MSCs after stimulation with a pro-inflammatory cytokine is greater than 70% when compared to MSC populations treated with a vehicle.

21. The method according to claim 17, further comprising assigning a grade to the potency of the MSCs based on the amount of produced anti-inflammatory molecules.
